(19) United States
(12) Patent Application Publication
(10) Pub. No.: US 2025/0042829 A1
(43) Pub. Date: Feb. 6, 2025

(54) METHANOL FROM BIOMASS GASIFICATION

(71) Applicant: CASALE SA, Lugano (CH)

(72) Inventors: Isabella MUSCIONICO, Orsenigo (IT); Pietro MOREO, Lugano (CH)

(21) Appl. No.: 18/718,112

(22) PCT Filed: Dec. 6, 2022
(86) PCT No.: PCT/EP2022/084537

§ 371 Date: Jun. 10, 2024

(30) Foreign Application Priority Data
Dec. 14, 2021 (EP) 21214547.8

(51) Int. Cl. C07C 29/151; C07C 29/80

(52) U.S. Cl. CPC C07C 29/1518; C07C 29/80

(57) ABSTRACT

Process for the synthesis of methanol comprising the steps of: subjecting a biomass to a gasification process in presence of steam and an oxidant; subjecting the so obtained gasifier stream to water gas shift conversion and purification to yield a synthesis gas with hydrogen, carbon monoxide and CO2; mixing said synthesis gas with a second stream of synthesis gas to yield a third stream of synthesis gas; feeding said third stream of synthesis gas to a methanol synthesis loop wherein a crude methanol and a tail gas retaining methane are produced; subjecting said tail gas to a reforming step in presence of an oxidant to generate said second stream of synthesis gas.

### FIELD OF APPLICATION

The invention is in the field of methanol production. The invention particularly pertains to a process and a plant for the synthesis of methanol.

### PRIOR ART

A gasification process involves the partial oxidation of a carbonaceous feedstock in presence of a sub-stoichiometric amount oxidant e.g. oxygen or air. Products of the gasification are a syngas comprising carbon monoxide, hydrogen and in a minor amount carbon dioxide, water and methane in addition to a solid residue that is not completely combusted or oxidized.

In the art there is a growing interest in using the syngas obtained from the gasification of biomass to generate bio-methanol. Biomass gasification is a complex process that needs to take into account conflicting requirements for instance the necessity to avoid the melting of the unburned residue and the necessity to keep the methane and carbon dioxide content in the syngas as low as possible.

Melting of the unburned residue must be prevented to avoid the transfer of impurities e.g. sulphur and alkali into the syngas. Said impurities may cause deactivation issues of the methanol synthesis catalyst and corrosion issues in the plant. Conversely, the formation of methane must be limited because said gaseous product does not participate in the synthesis of methanol.

Conventional biomass gasification processes are typically carried out in presence of oxygen and steam at a relatively low temperature e.g. below 1000° C. and at a medium to high pressure typically comprised between 30 to 100 bar.

Unfortunately, as a result of the operating conditions adopted i.e. low temperature and high pressure, the syngas obtained in said process retains a relatively high concentration of methane e.g. about 10-12% mol calculated on a dried basis.

The high quantity of methane retained in the syngas is a drawback because it penalizes the plant's productivity and efficiency as the unconverted methane is not exploited in the process but is typical purged from the synthesis loop as a tail gas and combusted.

An additional drawback that penalizes the cost and energy efficiency of the bio-methanol process concerns the utilization and the purification of the aqueous streams produced during the process.

In particular, methanol is obtained from the synthesis loop as a crude stream that contains impurities i.e. dissolved gases as methane, higher alcohols, aldehydes, ketones and water and must be purified downstream of the synthesis loop to obtain a high purity product. Purification is typically carried out in a distillation section wherein a high purity methanol product is separated from an aqueous stream contaminated with residual methanol and streams containing the above-mentioned impurities.

Said aqueous stream cannot be discharged directly into the environment but it must be treated into a suitable unit e.g. in a scrubber to reduce its CH3OH content. Obviously, said purification step increases the operational cost of the process and involves a waste of resources because the so obtained aqueous stream is subsequently discharged out of the plant.

Therefore, in light of the consideration stated above it is highly desirable to develop an energy-efficient, economically viable and resource-efficient process and plant for the synthesis of methanol.

US 2014/0145819 discloses a hybrid plant for liquid fuel production from hydrogen and carbon monoxide containing streams produced by gasifying solid carbonaceous feedstock and steam reforming of light fossil fuels.

### SUMMARY OF THE INVENTION

The aim of the present invention is to solve the drawbacks of the prior art.

The invention is based on the insight that in order to obtain a resource-efficient and energy-efficient bio-methanol process all the resources i.e. methane and aqueous streams produced during the synthesis are recycled in the process.

Accordingly, one aspect of the present invention is a process for the synthesis of methanol according to claim 1.

The process comprises the step of feeding a synthesis gas effluent obtained from the gasification of biomass and from the reforming of a tail gas to a methanol synthesis loop. In the methanol synthesis loop, catalytic conversion of carbon oxides to methanol is carried out under methanol synthesis conditions to obtain a methanol product and a tail gas rich in methane wherein said tail gas is partially recycled to the reformer for further conversion into a synthesis gas.

A further aspect of the present invention is a plant for producing methanol according to the claims.

The plant comprises a front end for producing a first stream of synthesis gas from the gasification of a biomass, a methanol synthesis loop for generating a crude methanol and a tail rich in methane and a tail gas treatment section comprising a reforming unit for converting the methane retained in said tail into a second stream of synthesis gas. The first stream of synthesis gas obtained from gasification and the second stream of synthesis gas obtained from reforming, when mixed, form a third stream of synthesis gas that is conveyed to the methanol synthesis loop.

The present invention provides an energy-efficient and economically viable option for producing bio-methanol because the methane that is synthesized as an unwanted product during the gasification can now be converted into a useful stream i.e. the second stream of synthesis gas in the reformer. The so obtained second stream of synthesis gas can then be converted into methanol to increase the productivity of the plant.

A further advantage is that an efficient reuse of the aqueous streams produced in the plant can be achieved and no methanol purification treatment of said aqueous stream is required. Still a further advantage is that no water is lost in the plant since the import and the export of water is balanced i.e. all the water produced is recycled in the process.

### DETAILED DESCRIPTION OF THE PRESENT INVENTION

The term biomass in the present invention includes but is not limited to wood material (bark, chips, scraps, and saw dust), pulp and paper industry residues, agricultural residues, organic municipal material, sewage, manure, and food processing by-products.

In the process of the present invention, said biomass feedstock is fed to a gasification step in presence of steam and an oxidant to generate a gasifier stream. The gasifier steam is then subjected to a water gas shift conversion and to a purification step to yield a first stream of synthesis gas retaining hydrogen and carbon monoxide.

The obtained first stream of synthesis gas is then mixed with a second stream of synthesis gas to yield a third stream of synthesis gas. The second stream of synthesis gas is obtained by subjecting a tail gas extracted from the methanol synthesis loop to a reforming process.

The third stream of synthesis gas is then conveyed to a methanol synthesis loop where it reacts catalytically to form crude methanol. In the methanol loop, a catalytic conversion of carbon oxides to methanol is carried out under methanol synthesis conditions, to yield a crude methanol and a tail gas retaining methane.

The tail gas extracted from the methanol synthesis loop is fed to a reforming step in presence of an oxidant to generate said second stream of synthesis gas that is then mixed with the first stream of synthesis gas obtained from the gasification unit.

According to a preferred embodiment, the biomass prior to be fed to the gasifier step is subjected to a series of pre-treatments to improve the efficiency of the gasification. Pre-treatments may include drying, pyrolysis and/or torrefaction.

Gasification is carried out in the presence of steam to increase hydrogen content in the synthesis gas and in the presence of an oxidant preferably oxygen. Oxygen can be produced on-site by means of an air separation unit or with a water electrolyzer. Preferably, the oxygen stream has a purity higher than 99% molar or preferably higher than 99.5% molar.

After gasification the syngas obtained may have the following composition: hydrogen content H2 comprised between 55 to 65% molar preferably equal to or of about 61.1% molar, a nitrogen content N2 comprised between 0.2 to 0.5% molar, preferably equal to or of about 0.4% molar, carbon monoxide CO content comprised between 22 to 28% molar, preferably equal to or of about 25.6% molar, carbon dioxide CO2 content comprised between 2 to 4% molar preferably equal to or of about 3.4% molar, methane CH4 content comprised between 8 to 10% molar, preferably equal to or of about 9.5% molar, Ar content comprised between 0.02 and 0.05% molar, preferably equal to or of about 0.04% molar.

The methanol synthesis catalysts are typically sensitive to tars, particulates, sulphur, excess of CO2 and other impurities like sulphur. For this reason, a gas cleaning section can be provided after the gasification step.

According to a particularly preferred embodiment, the gasifier stream leaving the gasification section can be subjected to a water gas shift conversion to adjust the H2/CO ratio.

The gas exiting the water gas shift conversion can be subjected to a cooling step prior to be conveyed to a CO2 removal step. The CO2 removal can be carried out via know process and technologies i.e. selexol, rectisol, MEA or MDEA chemical absorption.

According to the invention, the tail gas leaving the methanol synthesis section is subjected to a reforming step, preferably the reforming step is carried out under autothermal reforming conditions or under partial oxidation conditions in presence of an oxidant. Preferably, the reforming step is carried out at a temperature of 1000 to 1500° C., or 1000 to 1300° C.

Preferably the concentration of the inert gasses in the synthesis loop is less than 40% mol.

According to a particularly preferred embodiment the reforming step of the tail gas includes a pre-processing of the tail gas before the reforming reaction. Particularly preferably, the tail gas extracted from the methanol synthesis loop, prior to the reforming process, is subjected to a saturation step with water to obtain a saturated stream. More preferably the saturated stream is further contacted with steam to obtain a conditioned stream and said conditioned stream is conveyed to said reforming step. Preferably, said conditioned stream is characterised by a steam to carbon ratio S/C comprised between 1.0 and 2.0.

According to another particularly preferred embodiment, said crude methanol is subjected to a purification step to generate a methanol product, a first stream of fusel oil, a second stream of light end hydrocarbons and a recovered aqueous stream.

Preferably said recovered aqueous stream is supplied to said saturation step. Advantageously, all the contaminated water produced in the plant is recycled in the process and no additional water treatment is necessary to purify the water. Furthermore, an efficient usage of the resources is achieved.

Preferably, said conditioned stream obtained after mixing the saturated water stream with steam is further treated in a pre-heating stage to obtain a temperature-adjusted stream having a temperature comprised in the range 600° C. to 750° C., or preferably equal to 650° C. or about 650° C.

According to an embodiment of the invention, a further product of the methanol synthesis loop is a gas mixture of flash gas and tail gas and said pre-heating stage is carried out under direct firing conditions and is fired by said first stream of fusel oil, by said second stream of light-end hydrocarbons and by said gas mixture of flash gas and tail gas.

The term fusel oil is used hereinafter to indicate a mixture of heavier compounds comprising water, higher alcohols, methanol, and alkanes, Conversely, the term light-end hydrocarbons is used to indicate gaseous products that are lighter than methanol.

According to another embodiment of the invention, the pre-heating stage is carried out in a conventional heat exchanger or in an electrical heater and the temperature-adjusted stream is mixed with said first stream of fusel oil recovered from said purification step prior to be conveyed to said reforming step.

According to a particularly interesting application of the invention, said second stream of synthesis gas obtained from a reforming process prior to be mixed with said first stream of synthesis gas is subjected in sequence to a cooling step with the recovered aqueous stream generated in the purification step and to a separation step to condensate out a water condensate.

Preferably, said water condensate is mixed with said recovered aqueous stream from the purification step prior to exchange heat with said second stream of synthesis gas in said cooling step.

According to an embodiment, said cooling step comprises a steam generation step wherein said second stream of light-end hydrocarbons obtained from the purification section and said gas mixture of flash gas and tail gas obtained from the methanol synthesis loop are fired in said steam generation step to generate superheated steam. Preferably, firing conditions in the steam generation step are established by means of a fired heater.

Alternatively, superheated steam can be generated in said steam generation step by firing said first stream of fusel oil, said second stream of light-end hydrocarbons and said gas mixture of flash gas and tail gas.

Certain embodiments of the invention concern the use of various purge streams of the methanol synthesis process. Typically, a purge gas is extracted from the methanol synthesis loop, to avoid accumulation of inert gas and said purge gas, possibly after washing with water, is passed to a hydrogen recovery unit (HRU) to recover hydrogen contained therein, producing a stream of recovered hydrogen and a tail gas, named HRU tail gas. Said HRU tail gas may contain residual hydrogen and methane. Additional purge streams from the methanol process may include: flash gas from one or more separators, light ends and fusel oil from distillation of the crude methanol. In certain embodiments, at least a portion of said HRU tail gas is recycled as process gas to the reforming section. The other purge streams and optionally a remaining portion of said HRU tail gas can be recycled as fuel to a fired heater.

In an interesting embodiment of the invention, said HRU tail gas provides the majority of the process feed of said reforming step or more preferably the entire process feed of said reforming step. In the latter case, a considerable advantage of the invention is no need to additional fossil fuel for the reforming step. It can be said that the reforming step is performed in series relative to gasification step.

For example, according to embodiments of the invention, an autothermal reformer and a gasifier can be regarded as they operate in series because the process feed of the autothermal reformer is entirely provided by tail gas removed from a methanol synthesis process fed by the gasifier.

Preferably the HRU tail gas provides at least 80% or at least 90% or even more preferably 100% of the process feed of the reforming step.

Particularly preferably, the reforming step is performed in an autothermal reformer whose process feed is entirely provided by methanol tail gas. In an alternative embodiment, the reforming step is performed in a partial oxidation reactor.

The HRU tail gas may feed the reforming step after a proper treatment. Said treatment includes preferably saturation with water and may further include a pre-heating.

According to another interesting feature of the invention, the reforming step includes a pre-heating of the process stream to be reformed in a fired heater, and the fuel of said fired heater is at least partially provided by one or more of the above-mentioned purge streams, possibly with a small portion of the HRU tail gas.

Particularly preferably, said fired heater is fueled with said purge streams and, additionally, with a portion of the make-up gas of the methanol synthesis loop, which acts as a trim fuel to reliably control the combustion. More preferably, a stream of natural gas is used to feed pilot burners of the fired heater, for safety reasons.

In a preferred embodiment, the reforming step is performed in an autothermal reformer and the process feed at the inlet of said autothermal reformer contains at least 20% molar of methane on dry basis, preferably equal to or of about 50% molar of methane on dry basis.

In embodiments based on autothermal reforming, the reforming section preferably comprises an autothermal reformer as the only catalytic reactor in the reforming section.

According to the invention the plant comprises a front end including a gasification section configured for converting a biomass feedstock in presence of steam and an oxidant into a gasifier stream, a water gas shift converter and a CO2 removal section to yield a first stream of synthesis gas retaining hydrogen, carbon monoxide and residual CO2.

The plant further comprises a methanol synthesis loop, a tail gas treatment section and a line provided with a compression unit connecting said methanol front-end with said methanol synthesis loop.

The methanol synthesis loop includes a methanol synthesis reactor configured to generate a crude methanol and a tail gas.

The tail gas treatment section connects the methanol synthesis loop with said line connecting said methanol front-end with said methanol synthesis loop.

The tail gas treatment section includes a water saturation tower in communication with said methanol synthesis loop and configured to generate a saturated water stream, a pre-heating unit in communication with said saturation tower and a reforming unit in fluid communication with said preheating unit and configured to generate a second stream of synthesis gas.

The plant further comprises a line connecting said reforming unit with said compressor unit.

The plant may further include a line configured to supply steam to said saturated water stream prior to said pre-heating unit, and a purification section in communication with said methanol synthesis loop.

Preferably said purification section is a multi-column distillation section and is configured to generate a methanol product, a recovered aqueous stream, a first stream of fusel oil and a second stream of light-end hydrocarbons.

According to an embodiment, the plant furthers includes a line connecting said pre-heating unit with said purification section and said pre-heating unit is a fired heater and said reforming unit is an autothermal reformer.

Alternatively, the plant further includes a line connecting said purification section with said reforming unit and said reforming unit is a partial oxidation reactor.

Preferably, when the reforming unit is a partial oxidation reactor the pre-heating unit is a conventional heat exchanger or an electrical heater conversely when the reforming unit is an autothermal reformer the pre-heating unit is an electrical heater or a fired heater.

According to a different embodiment, when the pre-heating unit is a fired heater, the latter can be fired with said first stream of fusel oil, with said second stream of light-end hydrocarbons and with said gas mixture of flash gas and tail gas.

The plant preferably includes a cooling section comprising a steam generation section that is provided with a fired heater arranged downstream of said reforming unit and in fluid communication with said methanol synthesis loop and with said purification section.

The methanol synthesis loop preferably includes a methanol reactor provided with a fixed bed that operates in the pressure range of 50 to 120 bar and in the temperature range of 200 to 300° C.

### DESCRIPTION OF THE FIGURES

FIG. 1 represents a schematic process layout of a methanol plant according to a preferred embodiment of realisation of the invention.

FIG. 2 represents a schematic process layout of a methanol plant according to an alternative embodiment of the invention.

FIG. 3 represents a schematic process layout of a methanol plant according to another embodiment of the invention.

### DETAILED DESCRIPTION OF THE PREFERRED EMBODIMENTS

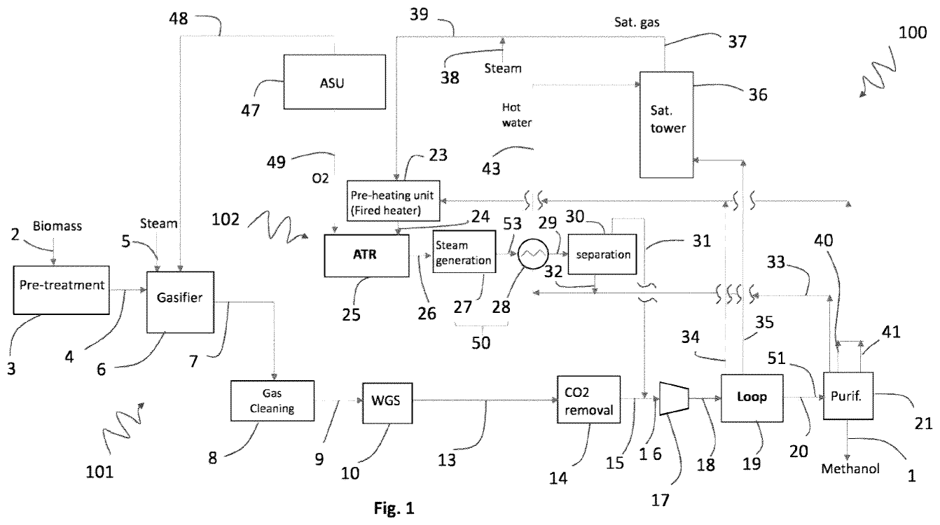

FIG. 1 shows a methanol plant 100 for the synthesis of methanol 1 comprising a front end 101, a methanol synthesis loop 19, a purification section 21 and a tail gas treatment section 102.

The methanol front end 101 comprises a pretreatment section 3, a gasifier 6, a gas cleaning unit 8, a water gas shift reactor 10 and a carbon dioxide CO₂ removal unit 14.

The tail gas treatment section 102 includes a water saturation tower 36 in communication with the methanol synthesis loop 19, a pre-heating unit and a reforming unit. In the embodiment of FIG. 1, the pre-heating unit includes a fired heater 23 and the reforming unit includes an autothermal reformer 25. The gas treatment section 102 further includes a cooling section 50 and a condenser 30.

An air separation unit 47 provides oxygen-containing stream 48, 49 to the gasifier 6 and to the autothermal reformer 25.

The methanol synthesis loop 19 and the purification section 21 are provided respectively with a methanol synthesis reactor and with a distillation unit (not shown). Said methanol synthesis loop 19 and said purification section 21 communicate between each other by means of line 51 that carry a crude methanol stream 20.

The methanol synthesis process is now elucidated with reference to FIG. 1. A biomass feedstock 2 is fed to the pre-treatment section 3 where the humidity of the feedstock 2 is reduced to a suitable level to yield a dried biomass 4.

The dried biomass 4 is fed to the gasifier 6 in presence of steam 5 and oxygen 48 to yield a gasifier stream 7. The gasifier stream 7 is then conveyed to a gas cleaning unit 8 where impurities such as sulphur and alkali are removed to generate a purified gas 9; the purified gas 9 is then fed to the water gas shift reactor 10 wherein the S/C ratio of the purified gas 9 is adjusted to a value suitable for methanol synthesis to yield an adjusted make-up gas 13.

The adjusted make-up gas 13 is then treated in the CO2 removal unit 14. Product of the CO2 removal unit 14 is a first stream of synthesis gas 15 that is mixed with a second stream of synthesis gas 31, which is extracted from the condenser 30, to yield a third stream of synthesis gas 16. Said third stream 16, suitable for conversion in a methanol synthesis loop, constitutes a methanol make-up gas.

The methanol make-up gas 16 is conveyed to a suction section of a compressor 17 to yield a compressed make-up gas 18 that is then fed to the methanol synthesis loop 19.

In the methanol synthesis loop 19 the crude methanol 20 is synthesized under methanol synthesis conditions. Other effluents of the methanol synthesis loop 19 are a tail gas 35 and a gas mixture 34 of flash gas and tail gas. Said tail gas 35 and said gas mixture 34 are both gaseous streams that retain methane. The tail gas in lines 34 and 35 may be taken from a hydrogen recovery unit processing a purge gas removed from the methanol synthesis loop.

The crude methanol 20 is then conveyed to the purification/distillation section 21 to generate the pure methanol product 1, a recovered aqueous stream 33, a first stream of fusel oil 40 and a second stream of light-end hydrocarbons 41.

The tail gas 35 is supplied to the saturation tower 36 where is saturated with hot water 43 to yield a saturated stream 37.

The saturated steam 37 is then contacted with steam 38 to generate a conditioned stream 39 that is preheated in the fired heater 23 and, after preheating, is fed to the autothermal reformer 25. Said stream 39 represents the entire feed of the autothermal reformer 25.

Said fired heater 23 is fired with said gas mixture 34 of flash gas and tail gas, with said first stream of fusel oil 40 and with said second stream of light-end hydrocarbons 41. Effluent of the fired heater 23 is a temperature-adjusted stream 24 that is then conveyed to the autothermal reformer 25.

Product of the autothermal reformer 25 is a reformed gas 26 that is fed in sequence to the cooling section 50 and to the condenser 30 to yield a water condensate 32 and said second stream of synthesis gas 31.

The second stream of synthesis gas 31, as above described, is then mixed with said first stream of synthesis gas 15 coming from the CO2 removal unit 14.

The cooling section 50 comprises a steam generation section 27 and one or more heat exchangers 28. The recovered aqueous stream 33 from the purification section 21 is supplied to the heat exchangers 28 to exchange heat with the cooled gas effluent 53 of the steam generation section 27. The heat exchangers 28 produce a hot water stream 43 that is conveyed to the saturation tower 36; the cooled gas stream 29 effluent from the heat exchangers 28 is fed to the condensation section 30.

As evident from the above-described embodiment all the resources generated in the process i.e. the tail gas 35 and the recovered aqueous stream 33 are recycled internally the process. In order to avoid the accumulation of impurities present in the water from the distillation, a purge stream can be discharged at the bottom of the saturation tower.

Advantageously, thanks to the above-described configuration, the applicant has discovered that the productivity of methanol can be increased by about 30% compared to a conventional bio-methanol process wherein the tail gas 35 and the recovered aqueous stream 33 are not recycled in the process.

In addition, the carbon efficiency calculated as mol of pure CH3OH in crude CH3OH/mol (CO+CO2+CH4) in the make-up gas is increased from 72% to 93%.

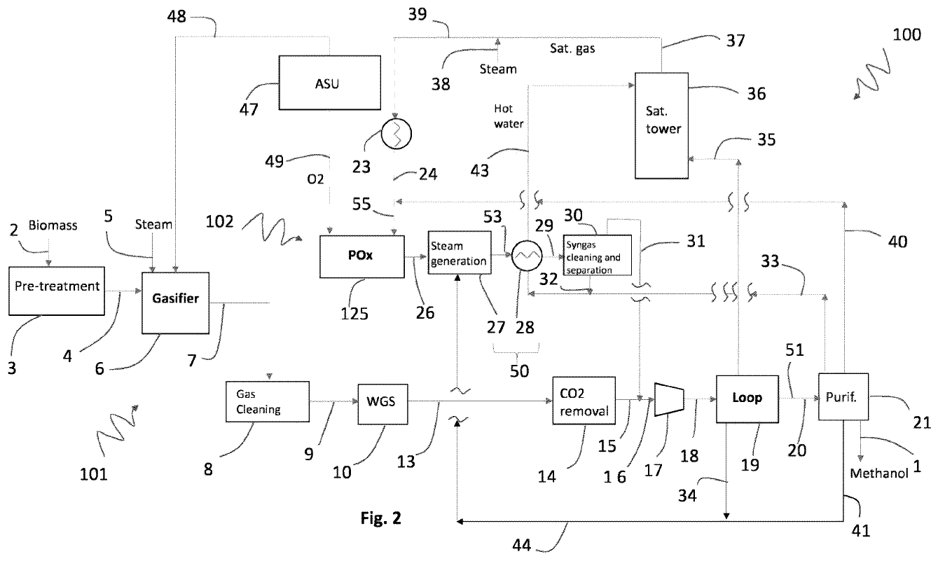

FIG. 2 shows a methanol plant 100 according to an alternative embodiment of the invention. This embodiment differs from FIG. 1 in that it uses a partial oxidation reactor (POX reactor) 125.

A first stream of synthesis gas 15 is synthesized according to the previously described steps. Herein, said first stream of synthesis gas 15 is mixed with said second stream of synthesis gas 31 and compressed in the compressor 17 to yield a compressed gas 18 that is then fed to the methanol synthesis loop 19.

Effluents of the methanol synthesis loop are a gas mixture 34 of flash gas and tail gas, a crude methanol stream 20 and a tail gas 35. The crude methanol stream 20 is fed to the purification section 21 to yield a methanol product 1, a recovered aqueous stream 33, a first stream of fusel oil 40 and a second stream of light-end hydrocarbons 41.

As in the previously described embodiment, the tail gas is supplied to the saturation tower 36 to yield a saturated gas 37 that is then mixed with steam 38 to yield a conditioned stream 39.

The conditioned stream 39 is then conveyed to a pre-heating unit 23 that in the present embodiment is represented by a conventional heat exchanger or an electrical heater to finally yield a temperature adjusted stream 24.

The temperature adjusted stream 24 is then mixed with said first stream of fusel oil 40 to yield a gas product 55 that is in sequence conveyed to the partial oxidation reactor 125.

Product of the partial oxidation is a reformed gas 26 that is then treated in a cooling step 50. The cooling step comprises a steam generation section 27 and heat exchangers 28. The steam generation section includes a fired heater (not shown in the figure) that is fired with a fuel gas 44 obtained by mixing said second stream of light end hydrocarbons 41 with said gas mixture 34 of flash gas and tail gas.

As in the previously described embodiment, the recovered aqueous stream 33 effluent from the purification section 21 is supplied to the heat exchangers 28 to indirectly exchange heat with a gas effluent 53 of the steam generation section 27.

Effluents of the heat exchangers 28 are a hot water stream 43 and a cooled stream 29. The hot water stream 43 is conveyed to the saturation tower 36 whilst said cooled stream 29 is conveyed to a syngas cleaning and condensation section 30.

Effluents of the condensation section 30 are a water condensate 32 and said second stream of synthesis gas 31. Said water condensate 32 is mixed with said recovered aqueous stream 33 that is then passed through the heat exchangers 28 to generate the hot water 43 the latter supplied to the saturation tower 36.

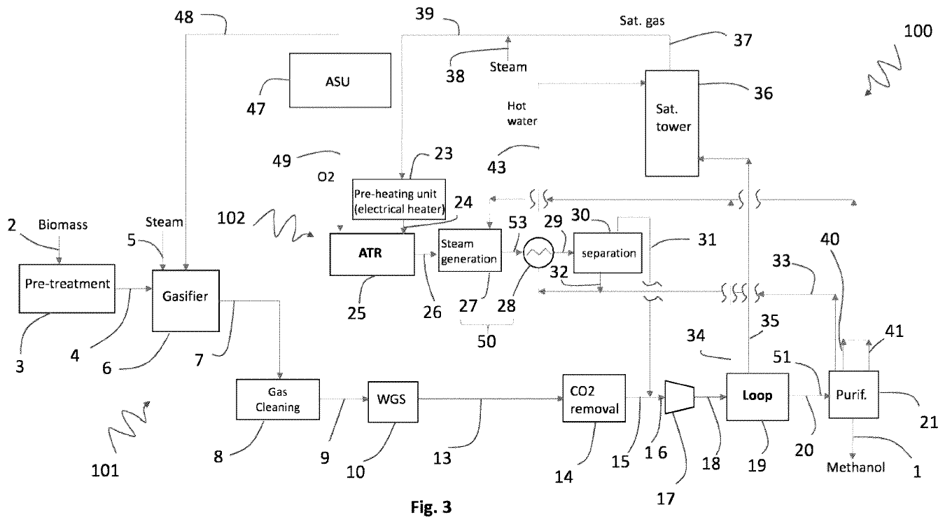

FIG. 3 shows an embodiment wherein the steam generation section 27 includes a fired heater that is fired with the gas mixture 34 of flash gas and tail gas and with the first stream of fusel oil 40 and the second stream of light-end hydrocarbons 41.

In further embodiments of the invention, the pre-heating unit 23, that is supplied with the conditioned stream 39, can be an electrical heater.

1-20. (canceled)

21. A process for synthesis of methanol, the process comprising:
a) subjecting a biomass feedstock to a gasification process in presence of steam and an oxidant to generate a gasifier stream;
b) subjecting said gasifier stream to a water gas shift conversion and to a purification step to yield a first stream of synthesis gas retaining hydrogen, carbon monoxide, and CO₂;
c) mixing said first stream of synthesis gas with a second stream of synthesis gas to yield a third stream of synthesis gas;
d) feeding said third stream of synthesis gas to a methanol synthesis loop in which catalytic conversion of carbon oxides to methanol is carried out under methanol synthesis conditions, thereby obtaining a crude methanol and a tail gas retaining methane; and
e) subjecting said tail gas to a reforming step in presence of an oxidant to generate said second stream of synthesis gas of step c).

22. The process according to claim 21, wherein said reforming step of point e) is carried out under autothermal reforming conditions or under partial oxidation conditions.

23. The process according to claim 21, wherein at least one of said oxidant of step a) or said oxidant of step e) is an oxygen stream having a purity higher than 99% molar.

24. The process according to claim 21, wherein step e) includes that the tail gas obtained in step d) is subjected to a saturation step with water to obtain a saturated stream and said saturated stream is further contacted with steam to obtain a conditioned stream and said conditioned stream is subjected to said reforming step.

25. The process according to claim 24, wherein said conditioned stream has a steam to carbon ratio S/C between 1.0 and 2.0.

26. The process according to claim 21, wherein said crude methanol is further subjected to a purification step to generate a methanol product, a first stream of fusel oil, a second stream of light-end hydrocarbons, and a recovered aqueous stream.

27. The process according to claim 26, wherein said recovered aqueous stream is supplied to said saturation step.

28. The process according to claim 24, wherein said conditioned stream prior to be fed to said reforming step is pre-heated in a pre-heating stage to obtain a temperature adjusted stream having a temperature in a range of 600 to 750° C.

29. The process according to claim 21, wherein a further product of the methanol synthesis loop is a gas mixture of flash gas and tail gas and said pre-heating stage is carried out under direct firing conditions and is fired by said first stream of fuel, by said second stream of light-end hydrocarbons and by said gas mixture of flash gas and tail gas.

30. The process according to claim 28, wherein said temperature adjusted stream is mixed with said first stream of fusel oil recovered from said purification step prior to be conveyed to said reforming step.

31. The process according to claim 21, wherein said second stream of synthesis gas of step c) prior to be mixed with said first stream of synthesis gas is subjected in sequence to a cooling step with said recovered aqueous stream generated in said purification step and to a separation step to condensate out a water condensate.

32. The process according to claim 31, wherein said water condensate is mixed with said recovered aqueous stream from the purification step prior to exchange heat with said second stream of synthesis gas in said cooling step.

33. The process according to claim 31, wherein said colling step comprises a steam generation step wherein said gas mixture of flash gas and tail gas, said second stream of light end hydrocarbons and optionally said first stream of fusel oil are fired in said steam generation step to generate superheated steam.

34. The process according to claim 21 wherein said tail gas provides the majority or the entirety of the process feed of said reforming step (e).

35. The process according to claim 21 wherein the reforming step includes a pre-heating of the process stream to be reformed in a fired heater, and the fuel of said fired heater includes one or more purge streams taken from the synthesis of crude methanol and/or purge streams taken from purification of crude methanol, and the fuel of said fired heater possibly includes one or more of: a portion of said tail gas; a portion of said third stream of synthesis gas of step d); or a stream of natural gas.

36. The process according to claim 21 wherein the reforming step (e) is performed in an autothermal reformer and the process feed at the inlet of said autothermal reformer contains at least 20% molar of methane on dry basis.

37. A plant for producing methanol, the plant comprising:
a) a front end including a gasification section configured to convert a biomass feedstock in presence of steam and an oxidant into a gasifier stream, a water gas shift converter and a CO₂ removal section to yield a first stream of synthesis gas retaining hydrogen, carbon monoxide and CO₂;
b) a methanol synthesis loop including a methanol synthesis reactor configured to generate a crude methanol and a tail gas;
c) a line provided with a compression unit connecting said methanol front-end with said methanol synthesis loop;
d) a tail gas treatment section connecting said methanol synthesis loop with said line connecting said methanol front-end with said methanol synthesis loop, wherein said tail gas treatment section includes:
a water saturation tower arranged downstream of said methanol synthesis loop and configured to generate a saturated stream;
a pre-heating unit in communication with said saturation tower;
a reforming unit in communication said preheating unit and configured to generate a second stream of synthesis gas; and
a line connecting said reforming unit with said compression unit.

38. The plant according to claim 37, further comprising:
a line configured to supply steam to said saturated stream prior to said pre-heating unit;
a purification section in communication with said methanol synthesis loop, said purification section includes a multi-column distillation section and is configured to generate a methanol product, a recovered aqueous stream, and a first stream of fusel oil and a second stream of light-end hydrocarbons.

39. The plant according to claim 38, further comprising:
a line connecting said pre-heating unit with said purification section and said pre-heating unit is a fired heater and said reforming unit is an autothermal reformer;
or
a line connecting said purification section with said reforming unit and said reforming unit is a partial oxidation reactor.

40. The plant according to claim 38, further comprising a cooling section including a steam generation section provided with a fired heater arranged downstream of said reforming unit and in fluid communication with said methanol synthesis loop and with said purification section.
